(19) United States
(12) Patent Application Publication
(10) Pub. No.: US 2023/0321185 A1
(43) Pub. Date: Oct. 12, 2023

(54) METHOD FOR TREATMENT OF TYPE-1 DIABETES IN A SUBJECT IN NEED THEREOF

(71) Applicants: Azam Moosavi, Karaj (IR); Mir Isa MOOSAVI, Karaj (IR)

(72) Inventors: Azam Moosavi, Karaj (IR); Mir Isa MOOSAVI, Karaj (IR)

(21) Appl. No.: 17/894,545

(22) Filed: Aug. 24, 2022

Related U.S. Application Data
(60) Provisional application No. 63/280,623, filed on Nov. 18, 2021.

(51) Int. Cl. A61K 38/17; A61K 38/27; A61P 3/10

(52) U.S. Cl. CPC A61K 38/1729; A61K 38/27; A61P 3/10

(57) ABSTRACT

A method for treatment of Type-1 diabetes includes the steps of administering manganin II peptide consisting of the amino acid sequence of SEQ ID 1 and administering recombinant human growth hormone.

### CROSS-REFERENCE TO RELATED APPLICATIONS

This application claims priority to the U.S. provisional patent application Ser. No. 63/280,623 filed on 18 Nov. 2021, which is incorporated herein by reference in its entirety.

The Sequence Listing XML that is contained in the file named “azam2.xml”, which is 1921 bytes (as measured in Microsoft Windows®) and was created on Jul. 4, 2023, is filed herewith by electronic submission, and is incorporated by reference herein.

Please replace any previously submitted sequence listing with the attached sequence listing.

### FIELD OF INVENTION

The present invention relates to a method for treatment of Type-1 diabetes, and more particularly, the present invention relates to the use of a combination of magainin peptides and human growth hormone in the treatment of Type-1 diabetes.

### BACKGROUND

Diabetes is a general term for heterogeneous metabolic disorders with chronic hyperglycemia as the main outcome due to disorder in insulin secretion or malfunction or both. According to the International Diabetes Federation (IDF) report, the number of diabetic patients from 138 countries was reported to be 463 million in 2019 (with an 8.3% outbreak). In addition, it is estimated that by 2030 and 2045, this number will rise to 578 million and 700 million, respectively.

Treatment of diabetes is currently a universal challenge due to the growing number of diabetic patients and high medication costs. In both Type-1 diabetes mellitus (T1DM) and Type-2 diabetes mellitus (T2DM), the two main types of diabetes, the progressive loss of functional β-cell mass and imbalanced blood glucose level, are the main common criteria. Therefore, the primary goal in the treatment of affected individuals is based on compensation of the endogenous insulin pool size via expanding the functional β-cell population, especially in T1DM.

At a glance, the pathophysiology of T1DM besides the effective role of environmental factors is related to evoking the immune system against β-cell antigens and beginning proinflammatory responses. β-cell antigens are presented to the immune system by antigen-presenting cells (APCs), then inefficient regulation of immune reactions induces chronic immunological responses that can result in β-cell destruction. The dendritic cells (DCs) then uptake the released antigens from destructed β-cells and present them to T cells. β-cells death through physiological or virus-directed mechanisms triggers the release of antigens and begins more immune responses against other β-cells. Dendritic cells (DCs) usually absorb these antigens and then present them to T-cells and induce autoreactive T-cells. Only when thymic negative selection does not recognize these autoreactive T cells, an auto-immune response is possible.

Activated autoreactive T cells by DCs stimulate autoreactive B and cytotoxic T cells. Finally, the effective mechanism of beta cell degradation requires the cooperation of DCs, T cells, B cells, natural killer (NK) cells, and macrophages. Based on the considerable number of destructed β-cells in T1DM, compensation of the functional β-cells population looks more promising treatment option.

Toward this goal, the maturation of various cell sources capable of differentiation, de-differentiation, and trans-differentiation have been evaluated. Many transcription factors have been identified that contribute to the differentiation of pancreatic epithelium, specification of endocrine progenitors, functional specialization of the α cells, and inducing of β and δ cells like Pdx1(7), Ngn3 (8), Arx, and Pax4. Among these transcription factors, the Arx and Pax4 have received more attention due to their roles in the final differentiation of β-cells. For instance, the ectopic expression of Pax4 can age-independently induce the continuous replacement of the embryonic glucagon-producing cells and their conversion into β-like cells. Furthermore, it has been shown that the expression of Pax4 in the mouse α-cells can result in the neogenesis or conversion of α-cells into functional β-like cells. Studies have also shown that Arx inactivation in pancreatic glucagon-positive cells can be transformed into β-like cells. It is interesting to note that, however, the function of Arx and Pax4 in the maturation process of endocrine cell lines have an inhibitory effect on each other's expression. Consequently, any therapeutic agent used to compensate for the loss of beta cells is expected to result in similar changes in these transcription factors with similar trends.

Among the reported antidiabetic agents under investigation, antimicrobial peptides have received extensive attention in the last decade. Magainin-AM2, as an orthologue of Magainin-2 from amphibians, is a cationic, amphipathic, α-helical antimicrobial peptide with 23 amino acids with the lowest hemolytic activity. The proposed mechanisms for the antidiabetic activity of Magainin-AM2 include cell membrane depolarization, increasing intracellular calcium content, and enhancing the release of GLP-1 from GLUTag cells followed by insulin-release from the treated cells. Although magainin has been frequently studied to treat T2DM models, there are no reports of using magainin in beta cell regeneration in T1DM models.

Thus, a need is appreciated for novel methods for the treatment of type-1 diabetes.

### SUMMARY OF THE INVENTION

The following presents a simplified summary of one or more embodiments of the present invention in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to novel methods for the treatment of type-1 diabetes.

It is another object of the present invention to provide secondary and complementary treatment for Type-1 diabetes in patients that have received pancreatic Graft

In one aspect, disclosed is a method for treatment of type-1 diabetes in patients in need thereof using a synergistic combination of Magainin and growth hormone for beta cell regeneration.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIGS. 1A-1C are graphical representations showing the comparative results of FBS (fasting blood glucose) and GTT (glucose tolerance test) among diabetic and normal treated mice relative to their relevant control groups; FIG. 1A shows a comparison of the mean percentage of fasting blood glucose changes; FIG. 1B shows a comparison of blood glucose mean at GTT defined intervals among treated and untreated normal mice, FIG. 1C shows a comparison of blood glucose mean at GTT defined intervals among treated and untreated diabetic mice.

FIG. 2A-2E shows a Western blot analysis of P-ERK, P-STAT5, P-S6 and PAX4 in treated normal and diabetic mice relative to their relevant control groups; FIG. 2A shows an Immunoblot of pancreatic P-ERK, PSTAT5, P-S6, PAX4 and GAPDH; FIG. 2B shows a comparative immunoblot analysis for pancreatic P-ERK; FIG. 2C shows a comparative immunoblot analysis for pancreatic PSTAT5; FIG. 2D shows a comparative immunoblot analysis for pancreatic P-56; FIG. 2E shows a comparative immunoblot analysis for pancreatic PAX4. (n=3-5, p<0.05, analysis of immunoblot results by fiji-java6 and prism 5); N1 (normal, GH treat), N2 (normal, Mag treat), N3 (normal, Mag+GH treat), N4 (normal, saline treat), D1 (diabetic, GH treat), D2 (diabetic, Mag treat), D3 (diabetic, Mag+GH treat), D4 (diabetic, saline treat).

FIGS. 3A-3D show the hematoxylin and eosin staining of pancreatic sections; FIG. 3A shows microscopic images with X40, X100 and X400 magnification from pancreatic tissue sections of normal treated and control, stained with hematoxylin and eosin, FIG. 3B shows microscopic images with X40, X100 and X400 magnification from pancreatic tissue sections of diabetic treated and control, stained with hematoxylin and eosin, FIG. 3C shows Quantitative fold comparison of the cell numbers per islet in each treated groups versus their matched control group, FIG. 3D shows a comparative analyses of the mean of cell numbers per island; (***p<0.001,**p<0.01, *p<0.05 using ANOVA test; all data depicted as mean±SEM (n≥3)); N1 (normal, GH treat), N2 (normal, Mag treat), N3 (normal, Mag−GH treat), N4 (normal, saline treat), D1 (diabetic, GH treat), D2 (diabetic, Mag treat), D3 (diabetic, Mag−GH treat), D4 (diabetic, saline treat).

FIGS. 4A-4E shows Magainin (Mag) and GH induce insulin+ and glucagon+ cell regeneration in pancreatic islets, all comparative results confirm the consecutive treatment with Mag and GH is more effective in beta cell mass compensation; FIGS. 4A and 4B show the immunohistochemical staining performed on pancreas sections, 1^{st} row glucagon+, 2^{nd} row insulin+, 3^{rd} row DAPI nuclei+, and 4^{th} row the merged image; FIG. 4A shows a normal treated (N1, N2, and N3) and their control C, D, and E represent quantitative immunohistochemical analyses results; FIG. 4C shows increased fold of insulin+/glucagon+/both cell count/pixel in normal and diabetic treated mice versus their matched control group; FIGS. 4D and 4E respectively show the comparison of the mean of insulin+ and glucagon+ cell count/islet of each group with result of other groups; (**p<0.01, ***p<0.001 using one-way ANOVA test, n=3); all data depicted as mean±SEM in C, D and E; N1 (normal, GH treat), N2 (normal, Mag treat), N3 (normal, Mag+GH treat), N4 (normal, saline treat), D1 (diabetic, GH treat), D2 (diabetic, Mag treat), D3 (diabetic, Mag+GH treat), D4 (diabetic, saline treat).

FIGS. 5A-5E illustrates Mag and GH induced ki67+ and vimentin+ cells of pancreas; all comparative results confirm consecutive treatment with Mag and GH is more effective in cell regeneration; FIGS. 5A and 5B show immunohistochemical staining performed on pancreas sections: 1^{st} row ki67+, 2^{nd} row vimentin+, 3^{rd} row DAPI labeled nuclei, and 4^{th} row the merged image; FIG. 5A illustrates results for normal treated (N1, N2 and N3) and their control (N4) mice; FIG. 5B shows the results of diabetic treated (D1, D2 and D3) and their control (D4); FIGS. 5C, 5D, and 5E represent quantitative immunohistochemical analyses results; FIG. 5C shows an increase fold of ki67+/vimentin+ pixel in normal and diabetic treated mice versus their matched control group; FIGS. 5D and 5E, respectively compare the percent of ki67+ and vimentin+ pixels of each group with result of other groups (**p<0.01, ***p<0.001 using one-way ANOVA test, n=3). All data depicted as mean±SEM in C, D and E. N1 (normal, GH treat), N2 (normal, Mag treat), N3 (normal, Mag−GH treat), N4 (normal, saline treat), D1 (diabetic, GH treat), D2 (diabetic, Mag treat), D3 (diabetic, Mag−GH treat), D4 (diabetic, saline treat).

FIGS. 6A-6E show that Magainin and GH induce the reduction of CD19+ and CD3+ cells in the pancreas; all comparative results confirm consecutive treatment with Mag and GH is more effective in attenuation of B and T cells that are involved in the beta cell degradation mechanism. FIGS. 6A and 6B show immunohistochemical staining performed on pancreas sections: 1st row is related to CD19+ cells, 2nd row CD3+ cells, 3rd row DAPI labeled nuclei, and 4^{th} row represents the merged image. FIG. 6A illustrates the results of a normal treated (N1, N2 and N3) and their control (N4) mice. FIG. 6B illustrates the results of diabetic treated (D1, D2 and D3) and control (D4) type 1 mice. FIGS. 6C, 6D and 6E represent quantitative immunohistochemical analyses results. FIG. 6C shows increase fold of CD19+/CD3+ pixels in normal and diabetic treated mice versus their matched result of other groups (*p<0.05, **p<0.01, ***p<0.001 using one-way ANOVA test, n=3). All data are depicted as mean±SEM in 6C, 6D and 6E. N1 (normal, GH treat), N2 (normal, Mag treat), N3 (normal, Mag+GH treat), N4 (normal, saline treat), D1 (diabetic, GH treat), D2 (diabetic, Mag treat), D3 (diabetic, Mag+GH treat), D4 (diabetic, saline treat).

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprise”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

Disclosed is a method for treatment of Type-1 diabetes in patients in need thereof. Disclosed is a method for treating Type-1 diabetes in patients in need thereof using a synergistic combination of Magainin and growth hormone. More specifically Magainin peptide is Magainin-II (23 aa) of SEQ ID No. 1. Magainin II is a cationic, amphipathic, a-helical antimicrobial peptide with twenty-three amino acids, and both the N-Terminus and C-terminus are free i.e., free acid and free amine. The method includes the steps of administering a combination of Magainin-II (SEQ ID No. 1) and growth hormone (SEQ ID No. 2) in a patient in need thereof. It is understood, however, that any terminal amine-derivative or terminal acid derivative including salts of carboxylic acid are within the scope of the present invention.

TABLE 1
Magainin-II

| Peptide | Sequence | No. |
|---|---|---|
| Magainin-II | GIGKFLHSAKKFGKAFVGEIMNS | SEQ ID No: 1 |

Treatments with the synergistic combinations of Magainin and hGH improve the results of Fasting blood sugar (FBS) and glucose tolerance test (GTT).

### Experiment 1

### Materials:

Chemicals: Streptozotocin, glucose, sodium dihydrogen phosphate, and tris sodium hydroxide were purchased from Sigma. Citric acid, sodium citrate, potassium tartrate sodium, sodium deoxycholate, acrylamide, methanol, ethanol and isopropanol were obtained from MERK, also from LKB, N,N′-Methylene base acrylamide, sodium dodecyl sulfate, bromophenol blue, Triton-X, TEMED, sodium chloride, and Dithiothreitol were prepared from SERVA.

Peptide Magainin II (GIGKFLHSAKKFGKAFVGEIMNS): It was synthesized by the Canadian Biomatik Company. The purification of this peptide (>95% purity) was performed by reverse phase chromatography using the Cromasil C18 column and the accuracy of this peptide was confirmed by mass spectrometry.

Human Growth Hormone (hGH): It was produced by EXIR company under the brand name of Exitropin 4 IU (1.3 mg) or (Somatropin), and their vials were purchased from a pharmacy in lyophilized powder form.

Laboratory animals: Male Balb/c mice, weighing about 25-30 g, were purchased from Razi institute (Karaj, Iran) and kept under standard conditions (an air-conditioned room (23±2° C.) with a 12 h light: 12 h dark cycle (light: 08:00-20:00 h)) and standard rodent diet in the animal house of the Institute of Biochemistry and Biophysics, University of Tehran. Also, all the processes of animal experiments were performed based on the national animal ethics committee guidelines.

Antibodies: mouse anti-p-ERK 1/2 antibody sc-81492 (1/1000), goat anti-PAX4 antibody (ab101721) (1/1000), rabbit anti-STAT5a antibody (ab30648) (1/1000), rabbit anti-GAPDH antibody (ab181602) (1/1000), rabbit anti-insulin antibody (ab63820) (1/500), mouse anti-glucagon antibody sc-514592 (1/500), mouse anti-vimentin antibody sc-6260 (1/500), rabbit recombinant anti-Ki67 (ab197547) (1/500), rat anti-CD3 antibody (ab11089) (1/500), mouse anti-CD19 antibody (sc-373897) (1/500). All secondary antibodies were utilized in at a 1/1000 concentration, including goat anti-rat IgG H&L (ab6840), goat anti-rabbit IgG H&L (ab6717), goat anti-rabbit IgG H&L (ab72465), goat anti-mouse IgG H&L (ab6785), goat anti-mouse IgG H&L (ab6787).

### Methods

### Study Design:

In a 14-day period, diabetes type 1 was induced in a group of mice via injecting multiple low doses of Streptozotocin (STZ). Also, the other group of mice received an equal volume of citrate buffer (pH 4.5) intraperitoneally (I.P.) in the same procedure that was called the normal group.

Each of the normal (N) (n=20) and diabetic (D) (n=20) groups was divided into four subgroups. The first group (N1 and D1) (n=5) received 6.7 mg/kg intraperitoneally growth hormone for two weeks. The second group (N2 and D2) (n=5) administrated 0.185 mg/kg intraperitoneally magainin II for approximately four weeks. The third group (N3 and D3) (n=5) first received 0.185 mg/kg of magainin II for four weeks, and then 6.7 mg/kg of growth hormone for two weeks. Finally, the fourth group (N4 and D4) (n=5) received only an equal volume of physiological saline instead of these treatments.

FBS measurements before the treatment and weekly during the treatment, and glucose tolerance test (GTT) at the end of the treatment were performed. The pancreatic tissue was quickly removed and placed in a cold phosphate buffer saline (PBS). Some of the extracted tissues for additional tests were stored in a freezer (−80° C.) and some were immersed in 4% paraformaldehyde (PFA) for immunohistochemical staining.

### Animal Maintenance and Manipulations:

All protocols especially animal maintenance and manipulation were conducted according to the guidelines of the animal ethics committee of the University of Tehran that were audited and accepted by this committee. Male Balb/c mice, weighing about 25-30 g, were purchased from Razi institute (Karaj, Iran) and housed in the animal laboratory of the institute of Biochemistry and Biophysics and maintained at 23° C.±2° C. with a 12-hour (8:00 to 20:00) light-dark cycle.

### Induction of Streptozotocin-Mediated Diabetes:

Diabetes type 1 was induced in a group of mice via injecting multiple low doses of Streptozotocin (STZ) (40 mg/kg, intraperitoneal) for five consecutive days. The STZ dissolved in the 50 mM sodium citrate buffer (pH 4.5) to a final concentration of 4 mg/ml, immediately prior to injection. Nine days after the last STZ injection, all the mice have their FBS (fasting blood sugar) over 200 mg/dl were considered as type 1 diabetic mice group.

### Glucose Tolerance Tests (GTTs) and Fasting Blood Sugar (Glucose) Measurement

FBS levels were measured after fasting mice for 6 h (7:00-13:00) with an Accu-Chek glucometer via tail vein. Also in the glucose tolerance test, six hours after the mice were deprived of food (7:00-13:00), the first FBS was measured and after that mouse administrated glucose (2 g/kg bodyweight, intraperitoneally), and then at the indicated time points post-injection, blood glucose levels were measured.

### Hematoxylin-Eosin Staining:

Fixed tissues in 4% paraformaldehyde, after embedding in paraffin, were cut in 6-μm sections, and the sections were applied to slides. For hematoxylin-eosin staining tissues respectively pass this process; rehydration incubation in hematoxylin (2.5 min), rinsing in water, dipping in 0.5% HCl/70% ethanol (v/v), washing in water, and again after immersion in 0.2% NaHCO₃, rinsing in water, dipping 20 secs in 0.1% eosin, washing briefly in water and finally dehydration and mounting.

### Immunohistochemistry:

Fixed tissue in 4% paraformaldehyde for 30 min at 4° C., embedded in paraffin and applied to slides. The prepared 6-μm sections were assayed as described previously. The following primary antibodies were used in these assays: rabbit anti-insulin antibody (ab63820) (1/500), mouse anti-glucagon antibody sc-514592 (1/500), mouse anti-vimentin antibody sc-6260 (1/500), rabbit recombinant anti-Ki67 (ab197547) (1/500), rat anti-CD3 antibody (ab11089) (1/500), mouse anti-CD19 antibody (sc-373897) (1/500). All secondary antibodies were utilized in at a 1/1000 concentration, including goat anti-rat IgG H&L (ab6840), goat anti-rabbit IgG H&L (ab6717), goat anti-rabbit IgG H&L (ab72465), goat anti-mouse IgG H&L (ab6785), goat anti-mouse IgG H&L (ab6787).

### Western Blot:

Proteins were extracted in RIPA buffer which are containing Tris-HCl buffer (100 mM, pH 7.5), ethylene diamine tetra acetic acid (EDTA, 10 mM), sodium pyrophosphate (10 mM), sodium fluoride (0.1 mM), sodium orthovanadate (10 mM), phenylmethylsulphonyl fluoride (PMSF, 2 mM), and aprotinin (10 mg/ml). The pancreas extracts were vortexed for 30 min frequently at 4° C. The homogenates were centrifuged at 12,000 rpm for 20 min at 4° C. The supernatants were stored in an −80° C. freezer. Total protein concentration was determined by the Lowry method.

De-freeze sample was added to loading buffer and dithiothreitol 0.5 M solution, then heated for 5 min at 95° C. Sodium dodecyl sulfate-polyacrylamide gel electrophoresis (SDS PAGE) and then protein transfer to a polyvinylidene difluoride (PVDF) blotting membrane was performed. Transferred PVDF membrane was nonspecifically blocked with fat free milk and after overnight incubation at 4° C. with primary antibody, (HRP)-conjugated secondary antibody was added and incubated for 120 min at room temperature. Finally, a result image was developed based on Western Blot-ECL (electrochemiluminescence) Development Protocol.

### Statistical Analysis:

All values are depicted as mean±SEM and considered significant if p<0.05. The arbitrary optical density unit was acquired using ImageJ software (version 1.46). Data were analyzed using GraphPad Prism software and the results of groups were compared with the ANOVA test.

The fasting blood sugars of mice in all diabetic and normal groups were measured before the treatment and weekly during the treatments. At the end of the treatments, the percentage of FBS changes were calculated. The average percentage of FBS alterations in each group was then determined. Finally, the average result of each group was compared and analyzed with other groups by prism software.

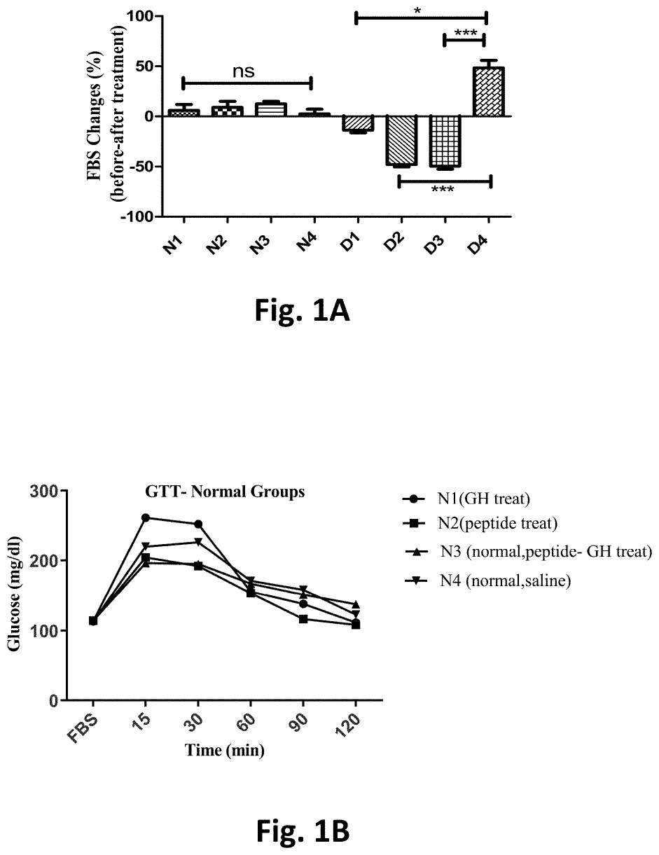

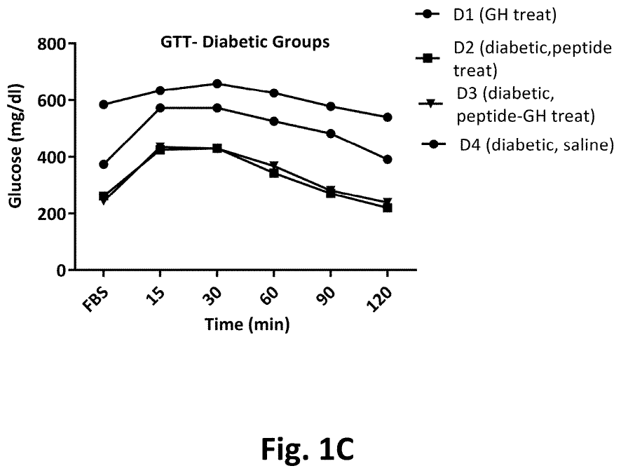

As shown in FIG. 1A, in normal mice, although the treatment had a slight increase in the mean percentage of FBS changes, this increase was not statistically significant. In contrast, significant changes were observed among the diabetic groups. In diabetic mice, with no hormone or peptide treatments (D4), the mean percentage of FBS changes increased by 49.23%, while the mean percentage of FBS changes in other diabetic groups decreased by 8.2, 47.9 and 49.6% for the treated mice by GH (D1), magainin (D2) and magainin then GH (D3), respectively.

Also, the approximate improvement rate of fasting blood sugar for treated diabetic groups in comparison with untreated control diabetic group, demonstrated 57.43%, 97.13% and 98.83% improvement in GH (D1), magainin (D2) and magainin then GH (D3) treated diabetic mice, respectively.

As evident from FIG. 1B, in normal mice, although injection of glucose solution raised blood sugar approximately in the first 30 minutes, the maximum increase occurred faster among the hormone- or peptide-treated groups within the first 15 minutes. Also, the maximum increase level in growth hormone-treated mice (N1) was higher than in other normal groups. Interestingly, the GTT chart pattern in a normal group with no treatment (N4) was observed in other groups and only in treated mice with magainin then GH (N3) blood sugar changes occur with a lower and smoother slope. Finally, after two hours, the blood sugar levels in all groups approached the normal values.

FIG. 1C shows the mean of blood sugar at defined times during the GTT in each diabetic group. Injection of glucose solution raised blood sugar in the first 30 minutes, although in the hormone or peptide treated mice (D1 and D2), this increase occurred in the first 15 minutes. Also, blood sugar levels among the untreated diabetic mice were higher than in other groups at all defined times and the interval changes in blood sugar were low and the glucose tolerance test curve in these mice showed no significant variation. The results of the glucose tolerance test were consistent with the results of the mean percentage of FBS changes in mice. For an instant, the growth hormone treatment (D1) had a less attenuating effect on blood glucose levels both in FBS and GTT than other treatments. Also, the results of FBS and GTT in mice treated with magainin and then growth hormone (D3) and magainin alone (D2) were similar and had the greatest effect on reducing blood sugar in mice.

GH and magainin cumulative effect on P-ERK level as an apoptosis reducer and pdx1 (differentiated beta cell factor) enhancer. Increased ERK and PDX1 expression could be survival for islet β-cells, the maintenance of islet cell mass, the cell proliferation and differentiation, and also the survival and pro-survival role for islet β-cells in tolerating gluco- or lipo-toxicity.

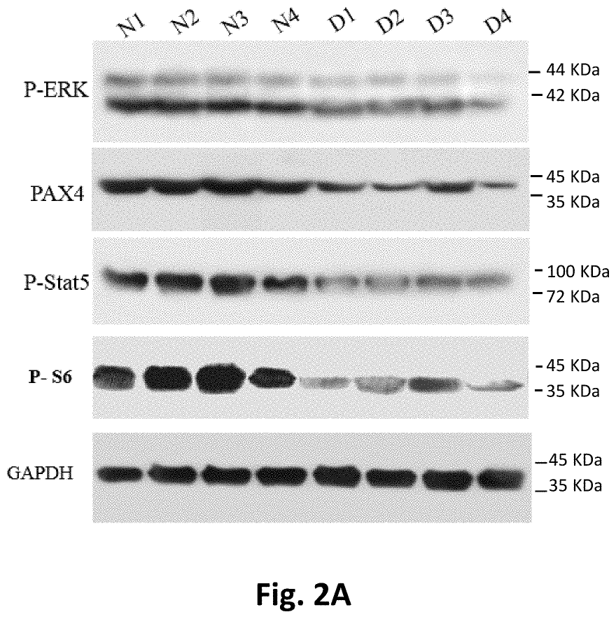

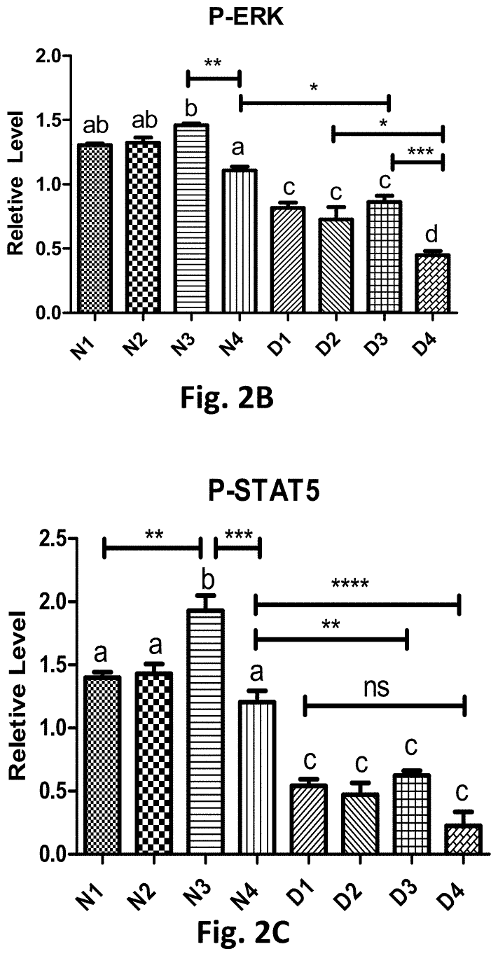

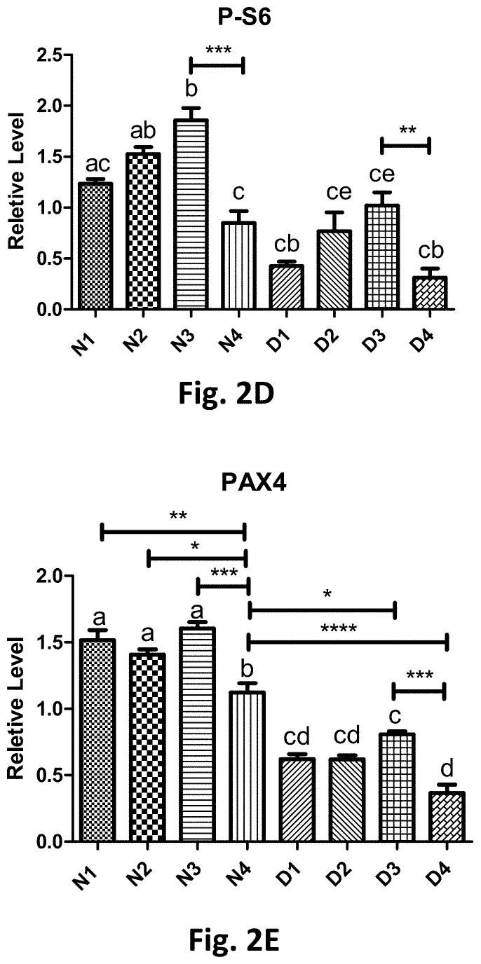

In this study, as shown in FIGS. 2A and 2B, the expression of the P-ERK factor in diabetic and normal mice has been investigated in comparison with their control group mice. In normal mice, although there was no significant difference in the P-ERK expression between mice treated with growth hormone (N1) and magainin peptide (N2), in normal treated mice with magainin and then growth hormone (N3), the P-ERK expression significantly increased by approximately 131% in comparison with the control group of the normal mice (N4). In diabetic mice, the P-ERK expression significantly increased by 191% for treated mice with magainin and growth hormone consecutively (D3). So based on the increased P-ERK expression, it was expected the partial inhibition of apoptosis, and PDX1 expression increase that can result in de-differentiation, trans-differentiation and consequently regeneration of functional beta-like cells which express insulin or glucagon or both of them.

### Induced P-STAT5 Level by GH and Magainin is Different in Normal and Diabetic Mice:

In this study as shown in FIGS. 2A and 2C, although a slight and non-significant increase in the expression of P-STAT5 was observed in all treated mice with growth hormone (N1) or magainin (N2) compared with the control group of normal mice (N4), the expression level of P-STAT5 was significantly increased by 161% in normal mice treated with magainin and then growth hormone (N3). Although the increased expression of P-STAT5 can be easily recognized in diabetic mice treated with growth hormone (D1) or magainin (D2) or consecutive treated with magainin and then growth hormone (D3) in comparison with the diabetic control group (D4), this enhancement is not statistically significant. It is noteworthy that the highest observed increase in P-STAT5 expression in diabetic mice was related to the treated mice with magainin and then growth hormone (D3) as observed in the normal groups.

GH and Magainin Cumulative Effect on mTOR Pathway can Induce Anabolic Process of Beta Cell Regeneration:

The activity of mTOR pathway was appraised by evaluating the changes in ribosomal P-S6 expression. As a downstream factor of the mTOR pathway, results of pancreatic P-S6 expression in all treated diabetic and normal mice were compared with their control groups. Therefore, changes in P-S6 expression proportionally reflects the activity of mTOR pathway. According to the FIGS. 2A and 2D, the P-S6 expression in normal mice treated with growth hormone (N1) or magainin (N2) were slight and not significant. However, in normal mice treated with magainin and then growth hormone (N3), the P-S6 expression approximately increased by 219% that in comparison with the P-S6 expression level in the normal control group was significant (N4). Diabetic mice demonstrated similar comparative results. Although there was no significant difference in the expression of P-S6 in mice treated only with growth hormone (D1) or only with magainin (D2), the P-S6 expression results in diabetic mice treated with magainin and then growth hormone (D3) demonstrated a significant 327% increase for the P-S6 expression in comparison with the diabetic control group (D4). It is noteworthy that the highest increase in P-S6 expression, as in the normal groups, was observed in the group treated with magainin peptide and then growth hormone (D3). Consequently, GH pulsatile secretion and temporally treatment period in this study via transient increase in mTOR activity can induce and enhance anabolic growth during the treatment, such as cell growth, ribosomal biogenesis, and protein synthesis.

### GH and Magainin Positive Role in Beta Cell Regeneration and Maintenance Via Enhancing PAX4 Expression:

Based on previous studies, stimulating the expression of Pax4 is essential for the a-to-β cell transdifferentiating strategy. Consequently, according to increased PAX4 expression in our treated groups, we have expected transdifferentiated or regenerated functional beta cells, especially in mice that have consecutively received magainin and then growth hormone, (N3, D3) and have significantly increased PAX4 expression.

FIGS. 2A and 2E show the expression of Pax4 in the treated and control mice. In normal groups, the expression of Pax4 in all treated groups was significant in comparison with the normal control group. In treated mice with growth hormone (N1), magainin (N2), and consecutively GH then magainin, the expression of Pax4 approximately increased by 135%, 125% and 143%, respectively. In diabetic mice, the expression level of Pax4 is totally lower than in normal groups. However, the Pax4 level in diabetic treated mice only with growth hormone (D1) or magainin (D2), demonstrated a similar but not significant increase, in mice with the consecutive treatment of magainin and then growth hormone (D3), in comparison with the diabetic control group (D4), the expression of Pax4 significantly increased by 221%. So based on these results, improvement in β-cells development and function is expected by enhancing Pax4 expression. The results were further confirmed by pancreas immunohistochemistry staining results.

The hematoxylin and eosin staining clearly demonstrated the effective role of magainin and growth hormone in islet size and average of cell numbers per islet. In hematoxylin and eosin staining, the islets number, and also cell numbers per islet in all sections were counted and the results were reported as the average number of cells per island for each group. Then, a comparative analysis was done for all treated normal and diabetic mice with their control groups.

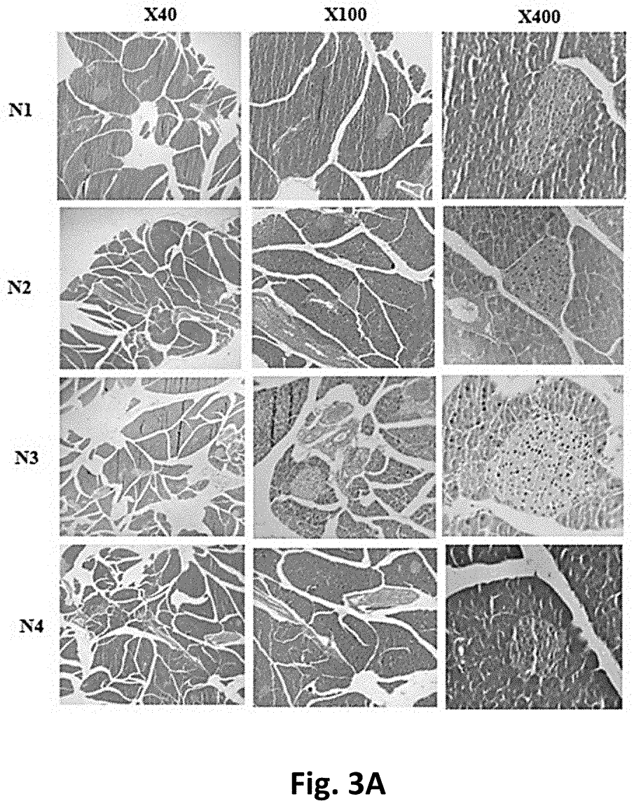

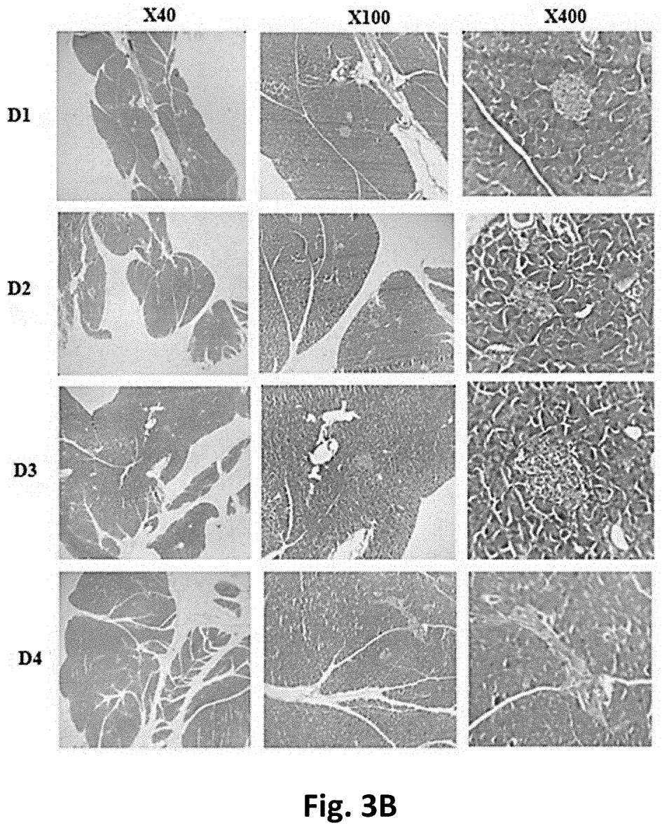

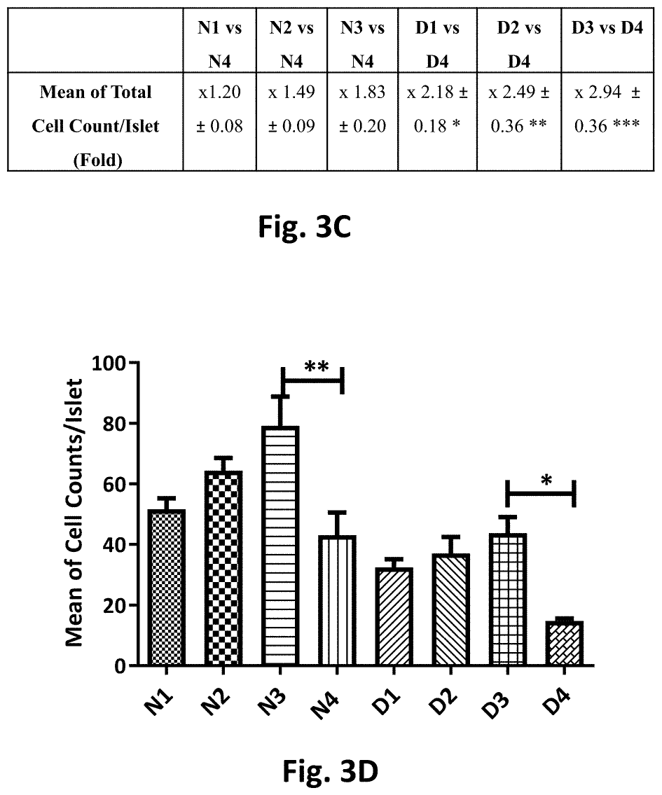

As shown in FIGS. 3A and 3B, microscopic images of hematoxylin and eosin staining at X40, X100 and X400 magnification have shown the positive effect of magainin and growth hormone on islet size and the number of islet cells.

FIG. 3C, shows the increased fold of cell count per islet in normal and diabetic treated groups versus their matched control group. In statistical comparison of increase folds for all treated groups, only diabetic treated groups have significant enhancement fold. Also, FIG. 3D quantitatively compares the mean of cell count/islet. For this part, all hematoxylin and eosin-stained pancreas sections has been quantitatively evaluated for average total cell count per islet. The average number of cells per islet only in the group of diabetic and normal mice treated with magainin and then growth hormone (D3 and N3), significantly increased by 294% and 184%, compared with their diabetic and normal control group (D4 and N4). In other groups that were treated only with peptide or growth hormone, despite a slight increase in the average number of cells per island, this increase was not significant compared to their control groups.

Magainin and growth hormone have a synergistic effect in beta cell mass compensation in STZ-induced diabetic mice. GH via growth hormone receptor (GHR) can affect glucose homeostasis, metabolism, growth, differentiation, and apoptosis in mammals. Also, in pancreatic beta cells, GH is effective in the cell cycle regulation, growth, and function of Langerhans Islands beta cells. On the other side, based on studies, magainin via enhancing GLP-1 release can induce insulin releasing and sensitivity and suppression of glucagon secretion in type 2 and obesity diabetic animal models. Also, Magainin-AM2 enhances insulin-releasing from mouse beta cell line via depolarization of cell membrane and enhancing intracellular calcium level. In this study, magainin and GH have been used to induce beta cell regeneration in type1 diabetes and almost all of the pancreatic beta cells have been eliminated by STZ.

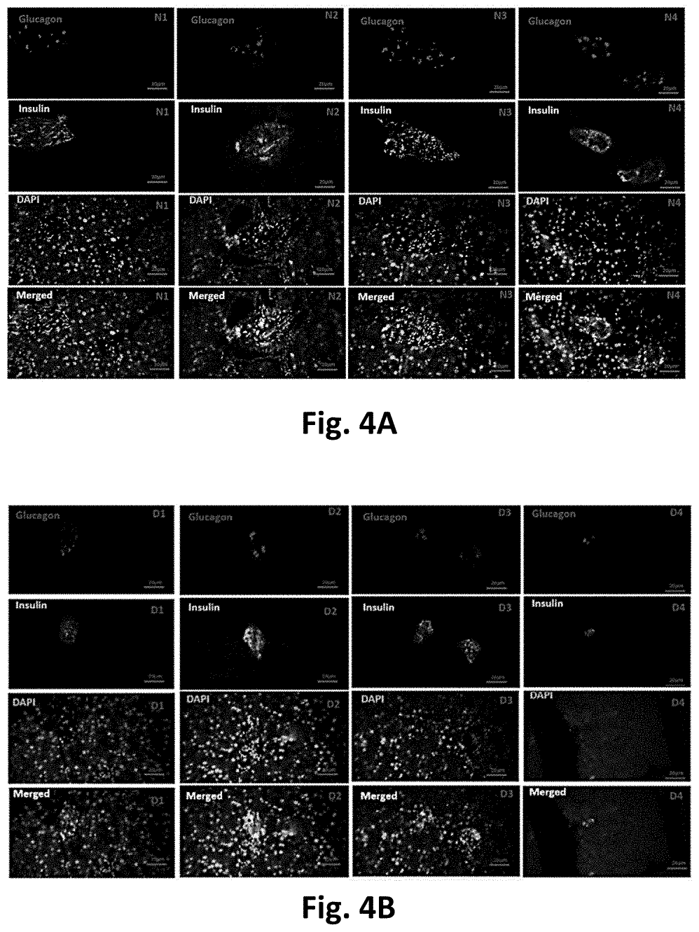

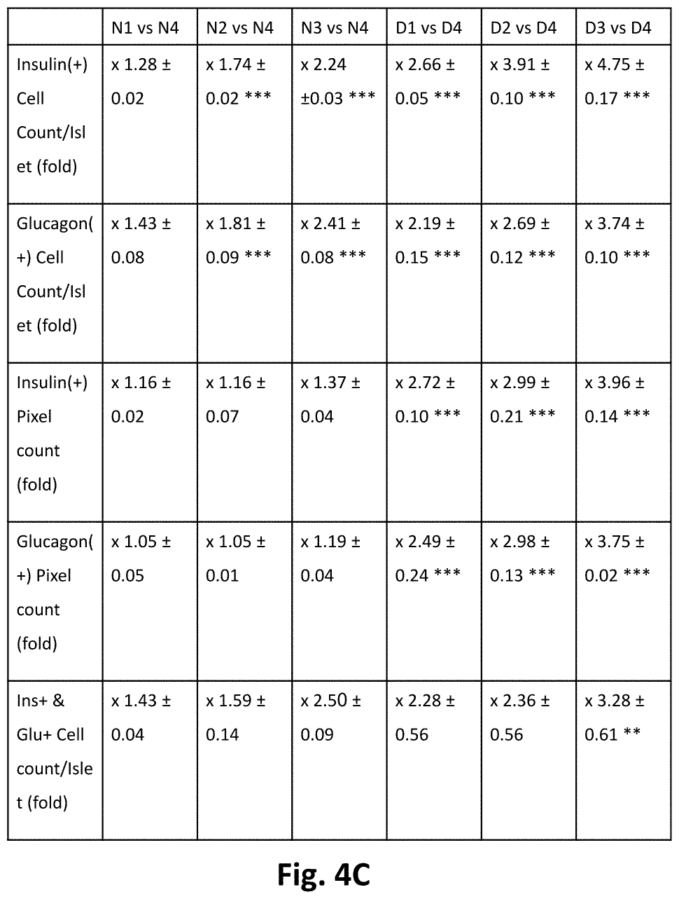

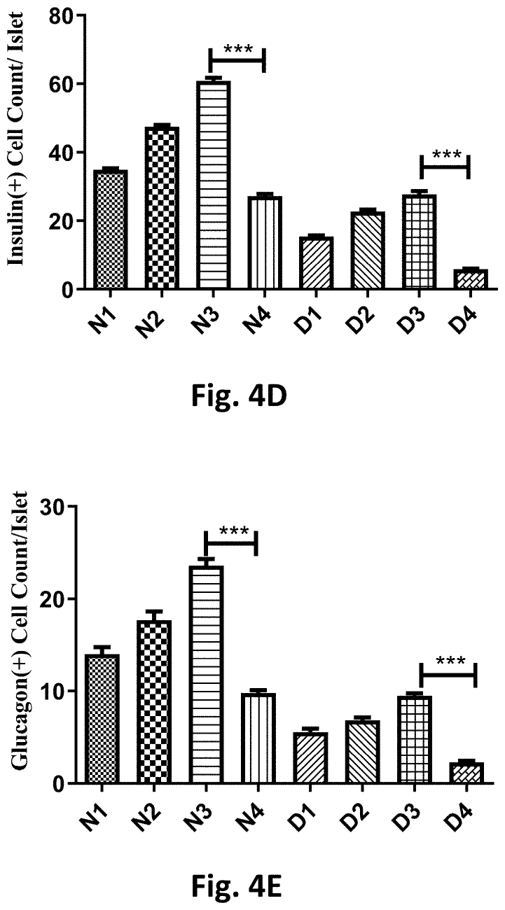

The examination of magainin, GH, and consequently magainin then GH treated mice demonstrated effective beta cell regeneration in type 1 diabetic mice. As shown in FIG. 4, all comparative results confirm consecutive treatment with magainin, and GH (N3 and D3) is more effective in beta cell mass compensation. In FIGS. 4A and 4B respectively related to normal and diabetic mice, immunohistochemical staining of one pancreatic section of each group has been shown on pancreas sections. Each column is related to one section belonging to a group. 1st row glucagon+, 2nd row insulin+, 3rd row DAPI labeled nuclei+, and 4th row merged of three previous pictures together. These pictures could be useful for qualitative comparison.

FIGS. 4C, 4D, and 4E represent quantitative immunohistochemical analyses results. FIG. 4C, shows an increase fold of insulin+, glucagon+, or both based on cell or pixel count in normal and diabetic treated mice versus their matched control group. Most of the calculated fold increase in treated diabetic mice are significant, except for co-expressed insulin and glucagon cells in D1 and D2. Co-expressed insulin and glucagon cells as a criterion for α to β-like cell trans-differentiation have been counted and evaluated by a skilled pathologist but more advanced methods are needed for absolute confirmation. Also, although increased values in normal treated groups are remarkable, only enhanced fold of insulin+ and glucagon+ cell numbers in N2 and N3 groups are significant. FIGS. 4D and 4E, respectively compare the mean of insulin+ and glucagon+ cell count per islet of each group with the result of other groups. This comparison used to improve the conception of quantitative beta cell regeneration based on percent or fold increase evaluations, especially in the control diabetic group.

### Effect of Magainin and Growth Hormone on Cell Regeneration and Proliferation:

In this study, Ki67 and vimentin were respectively used as a marker of cell division and differentiation of epithelial cells to mesenchymal stem cells in immunohistochemical staining of pancreatic tissue sections. Ki67 is actually an antigen that is found in the nucleus at all active stages of cell division (G1, S, G2 and mitosis); and the highest level of ki67 expression is related to the S phase of the cell cycle. However, this protein is not seen in the G0 phase (differentiation or cessation of the cell cycle) from the cell cycle. On the other hand, vimentin is a type of cytoskeletal protein that is specifically expressed in mesenchymal cells. This factor is a marker of differentiation of epithelial cells into mesenchymal stem cells (EMT) that these mesenchymal stem cells have the ability to differentiate into different types of cells.

In FIGS. 4A and 4B, immunohistochemical staining of the pancreas sections has been demonstrated, each column shows a section of the pancreas belonging to one group. The first row contains Ki67+ cells, the second row contains Vimentin+ cells, the third row DAPI-stained cell nuclei, and the fourth row contains the merged image. The qualitative and ocular comparisons demonstrate that magainin and growth hormone increase ki67+ and vimentin+ cells in the pancreas, and this increase appears to be greater in consecutive treatment of magainin and growth hormone in groups N3 and D3.

Due to the activation of multiple pathways in this treatment, the expression comparison of Ki67 and vimentin in pancreatic tissue sections is helpful to realize that the compensation of beta cells in these treatments depend on cell proliferation or regeneration of cells.

All comparative results confirm that consecutive treatment with magainin and growth hormone has a greater effect on increasing proliferation as well as increasing differentiation from mesenchymal cell lines in comparison with three control groups. For example, in FIG. 4C, in groups D3 and N3 that have been compared to their control groups, Ki67 expression multiplied by an average of 1.67 and 1.55 times, respectively, as well as the average expression of vimentin increased by 2.21 and 1.49 times, respectively. Regarding comparison results, it is reasonable that in mice with type 1 diabetes, the beta cells have been almost completely lost before the initiation of the treatment, and the rate of reproduction from the precursor cell lines increases more effectively.

The effect of magainin and growth hormone in the treatment of type 1 diabetes suggests it as a secondary treatment for transplant recipients

Although the pathophysiology of T1DM is related to environmental factors, evoking the immune system against β-cell antigens, and beginning proinflammatory responses, the effective mechanism of beta cell degradation requires the collective cooperation of DCs, T, B and natural killer (NK) cells and also macrophages. CD3 and CD19 are known as T and B lymphocyte surface antigens, respectively. So increased expression of these two factors indicates an increase in the distribution of immune T and B cells.

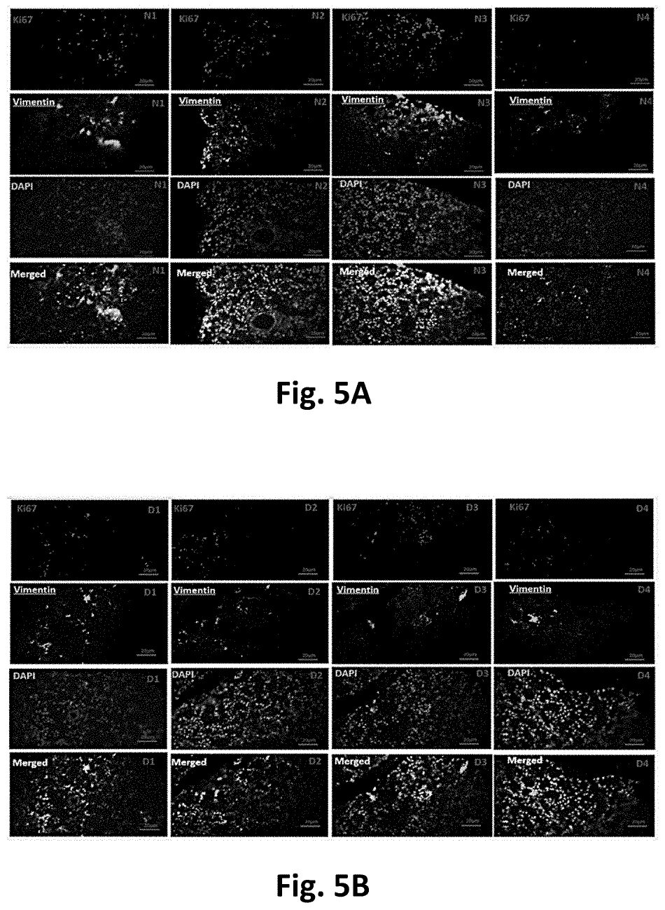

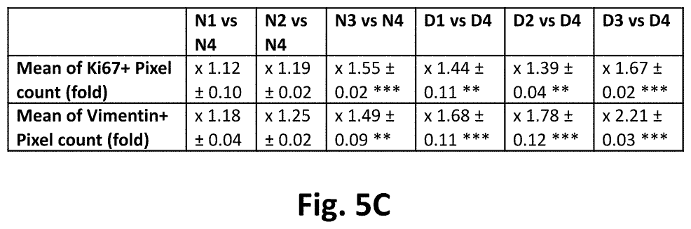

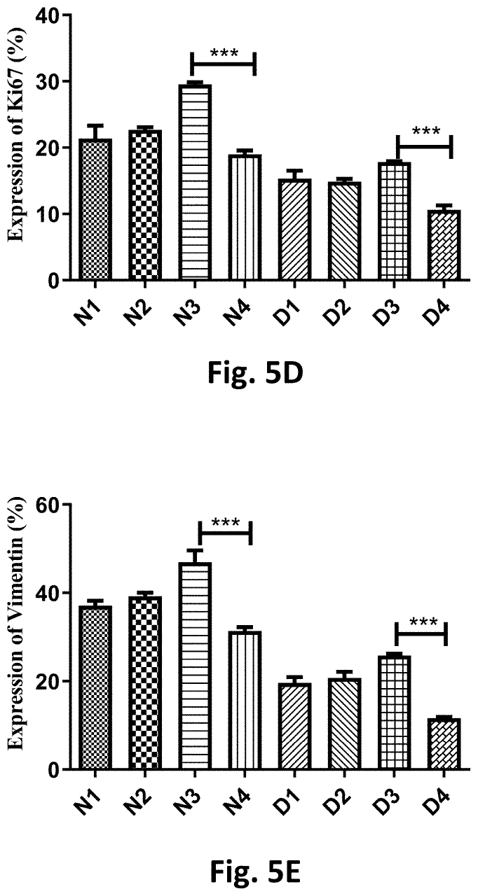

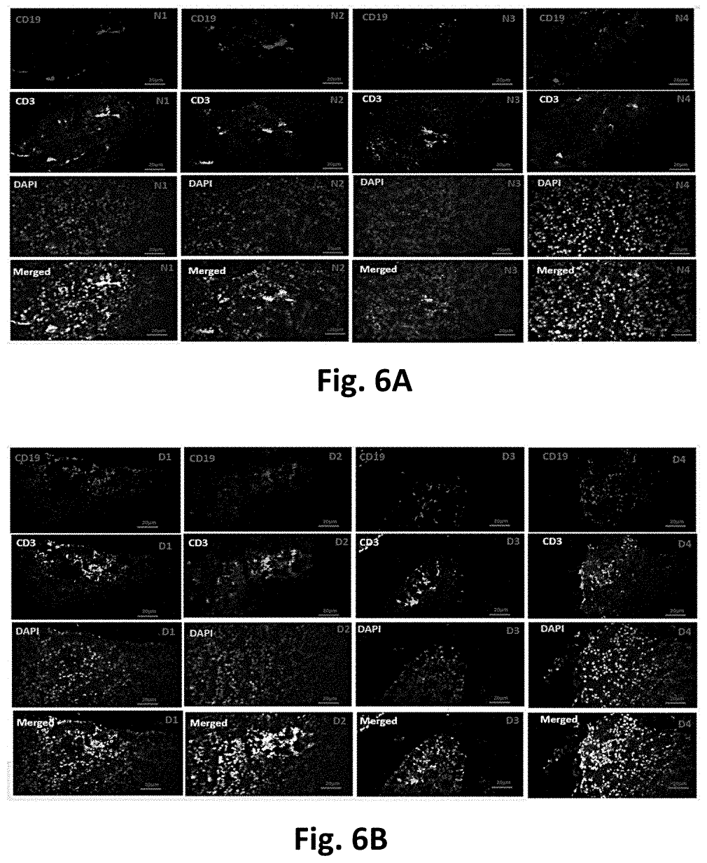

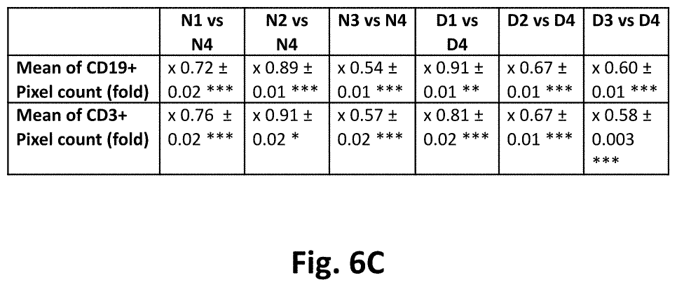

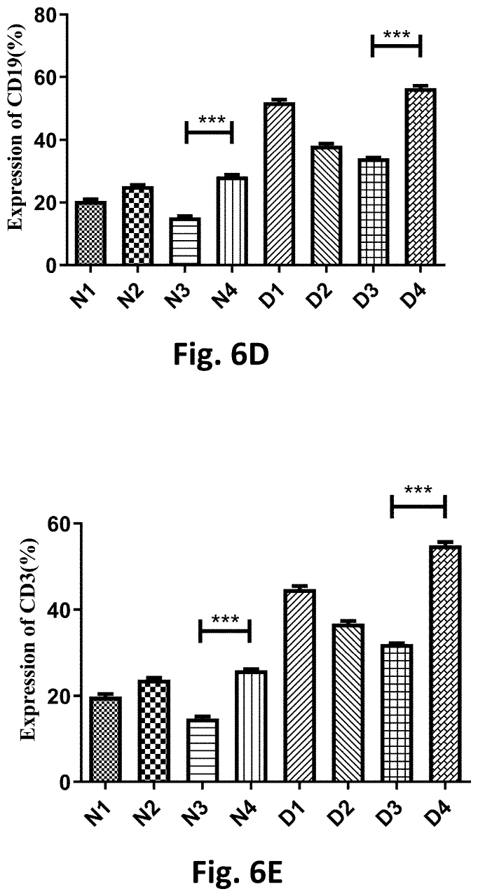

Referring to FIGS. 5A-5E, diabetic mice without any treatment have the most percentage of CD3+ and CD19+ pixels among all groups. All comparative results confirm consecutive treatment with magainin, and GH is more effective in the decrease of B and T cells that are involved in beta cell degradation mechanism. In FIGS. 6A and 6B, immunohistochemical staining representative pictures, the first row contains CD19+ cells, the second row contains CD3+ cells, the third row DAPI-stained cell nuclei, and the fourth row contains the merged image. The qualitative comparison of FIGS. 6A and B, demonstrate that magainin and growth hormone decrease CD19+ and CD3+ cells in the pancreas. The amount of CD3+ and CD19+ pixels effectively have decreased by 60 and 58 percent in D3 mice and also 57 and 54 percent in N3 mice respectively that consecutively have been treated with magainin and growth hormone (demonstrated in FIGS. 5C, 5D and 5E). Consequently, in a patient with type 1 diabetes that has received pancreatic graft, this kind of treatment on one side can improve and accelerate beta cell compensation and on the other side due to T and B cell number reduction, new beta regenerated cells can be more faithful.

### Discussion and Conclusion:

Here, disclosed is an unsuspected role of magainin in beta cell regeneration that was confirmed based on hematoxylin and eosin staining that clearly demonstrated regeneration of islet cells and reconstruction of islets in type 1 diabetic mice and also a more effective role of consecutive treatment with magainin and growth hormone in islet size and the average of cell numbers per islet in both normal and diabetic mice. Improved FBS and GTT results in treated diabetic mice, demonstrated that regenerated cells including functional beta cells that magainin can induce their insulin secretion more effectively.

Furthermore, IHC staining analysis approved insulin+ cell regeneration increased up to approximately 5-fold, although glucagon+ cell enhanced significantly, the ratio of insulin positive cell versus glucagon positive cell preserved in a normal range. Also on one side, based on increased PAX4, we expected more alpha to beta cell trans-differentiation and increased cell differentiation that resulted in differentiated beta cells. On another side, according to the semi-quantitative investigation performed on IHC staining and counting analysis, co-expression of insulin and glucagon in D3 was found to be significantly increase which could be evidence of enhancement in differentiation and trans-differentiation.

Due to the evaluation results of the activation of multiple pathways in this study, the effects of these treatments have been simplified in FIG. 5. As shown in FIG. 5, magainin increases insulin secretion and inhibits the apoptotic pathway by increasing intracellular calcium. Also, magainin can enhance the expression of the insulin gene by increasing the levels of GLP-1. On the other hand, GLP-1 can inhibit beta cell dedifferentiation via FOXO1. Furthermore, this increase in GLP-1 also indirectly increases ERK-1,2 and p-S6 (AKT), and consequently can indirectly inhibit the apoptosis pathway by them. Magainin also increases the expression of some cell cycle and anti-apoptotic genes.

In addition, growth hormone also acts by increasing the amount of ERK1,2 and (p-S6) AKT and STAT5 and also increases intracellular calcium through the prolactin receptor (PRLR), and consequently increasing insulin secretion and inhibitory effects on the apoptotic pathway.

Furthermore, Pdx1 is an important transcription factor in differentiating progenitor cell lines into pancreatic endocrine cells such as beta cells. Based on a study, the increase in Pdx1 was associated with an increase in ERK. In other words, Pdx1, as a downstream gene regulated by ERK, had increased directly or indirectly under the influence of ERK1,2. Also as another transcription factor PAX4 acts as a determining factor in the differentiation of endocrine cells into beta and delta cells. under the influence of PAX4, beta and delta cells differentiate, or some alpha cells can trans-differentiate into beta cells. In general, magainin and growth hormone can compensate beta cells by the de-differentiation pathway, directly and indirectly, inhibiting apoptosis, inducing alpha cell trans-differentiation into beta cell and also progenitor cell differentiation into beta cell.

Regarding these approximately successful results probably the disclosed treatment can be also effective in pancreas implant cases, in which there are some normal cells to accelerate regeneration more effectively. As another suggests, the disclosed treatment strategies can be adjusted based on personal medicine to get better remedies.

In certain implementations, the disclosed growth hormone can be Somatropin which is a purified polypeptide of recombinant human growth hormone, for example commercially available Exitropin®. The dose of Somatropin in combination with Magainin II can be commercially determined, for example, the dose of growth hormone can be about 4 IU i.e., 1.3 mg.

In certain implementations, the effective dose based on previous research and IC50 results were determined: The normal (N, n=20) and diabetic (D, n=20) groups were each divided into four subgroups. The first group (N1 and D1, n=5) received (IP) 6.7 mg/kg/day of growth hormone for 14 days. The second group (N2 and D2, n=5) were daily administrated 0.185 mg/kg magainin II (IP) for approximately 28 days. The third group (N3 and D3, n=5) first received 0.185 mg/kg magainin II for 28 days and then 6.7 mg/kg growth hormone for 14 days. Finally, the fourth group (N4 and D4, n=5) received only an equal volume of physiological saline. In this study administration of solved lyophilized powder has done intraperitoneally (IP), but in the developing phase drug delivery based on personalized medicine can multiply effectiveness. Thus, it is understood that all modes of drug administration including parenteral and oral are within the scope of the present invention. Also, different dosage forms, such as tablets, capsules, transdermal patches, and the like are within the scope of the present invention. Also, due to the possibility of insulin hypersensitivity, the third group first received 0.185 mg/kg magainin II for 28 days and then 6.7 mg/kg growth hormone for 14 days. Combined administration of Magainin and GH was found to have a synergistic effect, more preferably, the consecutive administration of Magainin followed by GH was found to be more effective to treat type 1 diabetes. The treatment with magainin peptide and growth hormone can be used as a primary treatment due to significantly induce regeneration of beta cells in type 1 diabetes. Treatment with magainin peptide and growth hormone can significantly reduce T and B cells, so can prevent beta cell destruction during the treatment and as secondary treatment in pancreas transplantation to accelerate cell proliferation and differentiation and reduce immune responses.

According to the treatment strategy, treatment with magainin peptide and growth hormone in diabetic and normal mice (groups N3 and D3 in this study) and a significant decrease in CD3+ and CD19+ cells in these groups, also due to a temporary increase in the mTOR pathway (temporary immunosuppressant), this treatment can be used as an adjunctive or secondary treatment in pancreatic transplantation of type 1 diabetes, kidney transplantation and other transplants in order to increase transplantation efficiency and reduce the level of immune responses in transplant rejection.

Considering the action mechanism of magainin and growth hormone, the growth factors including GLP-1, IGF, EGF and etc., can show similar effects with magainin. According to the treatment strategy, treatment with magainin peptide and growth hormone in diabetic and normal mice (groups N3 and D3 in this study) has a synergistic effect on signaling pathways, which is confirmed by a significant increase in P-ERK and P-S6. It can pave the way for the widespread use of this treatment in other diseases, disorders and transplants (In the case of diseases or complications due to decreased function of the mTOR pathway, such as sarcopenia, aging, or other diseases with a similar mechanism, this treatment can be used, with the effect that this pathway has on glucose, fat, brain function and it has muscle as well as muscle mass, it can reduce complications or improve function).

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A method for treatment of Type-1 diabetes in a patient in need thereof, the method comprising the steps of:
administering manganin II peptide consisting of amino acid sequence of SEQ ID 1; and
administering recombinant human growth hormone.

2. The method according to claim 1, wherein the recombinant human growth hormone is Somatropin.

3. The method according to claim 1, wherein the recombinant human growth hormone is administered upon administering the manganin II peptide.

4. The method according to claim 3, wherein the recombinant human growth hormone is administered after a predetermined period upon administering the manganin II peptide.

5. The method according to claim 4, wherein the recombinant human growth hormone and the manganin II peptide are administered intraperitoneally.

6. A composition for treatment of Type-1 diabetes in a patient in need thereof, the composition comprising:
manganin II peptide consisting of amino acid sequence of SEQ ID 1; and
a recombinant human growth hormone.

7. A parenteral dosage form for treatment of Type-1 diabetes in a patient in need thereof, the parenteral dosage form comprises:
manganin II peptide consisting of amino acid sequence of SEQ ID 1; and
a recombinant human growth hormone.

8. The parenteral dosage form according to claim 7, wherein the parenteral dosage form is intraperitoneal dosage form.


SEQUENCE LISTING (1 sequence)

SEQ ID NO: 1 - protein, 23 aa, Xenopus laevis
GIGKFLHSAKKFGKAFVGEIMNS